(19) United States
(12) Patent Application Publication
(10) Pub. No.: US 2023/0034483 A1
(43) Pub. Date: Feb. 2, 2023

(54) DEFORMABLE RETINAL ELECTRODE DEVICE

(71) Applicant: KOREA UNIVERSITY RESEARCH AND BUSINESS FOUNDATION, Seoul (KR)

(72) Inventors: Seong Woo KIM, Seoul (KR); So Hee KIM, Daegu (KR); Hee Won SEO, Gyeonggi-do (KR); Nam Ju KIM, Seoul (KR)

(73) Assignee: KOREA UNIVERSITY RESEARCH AND BUSINESS FOUNDATION, Seoul (KR)

(21) Appl. No.: 17/759,003

(22) PCT Filed: Jan. 21, 2021
(86) PCT No.: PCT/KR2021/000821

§ 371 Date: Jul. 18, 2022

(30) Foreign Application Priority Data
Jan. 23, 2020 (KR) 10-2020-0009518

(51) Int. Cl. A61N 1/05; A61N 1/36

(52) U.S. Cl. CPC A61N 1/0543; A61N 1/36046

(57) ABSTRACT

A retinal electrode device according to an embodiment comprises: a base, a first wing connected to a first side part of the base, wherein a first folding line is defined between the base and the first wing, and a plurality of electrodes installed on at least one of the base and the first wing, wherein the retinal electrode device may be configured to have a first form in which the first wing is folded on the base along the folding line and a second form in which the first wing is unfolded on the base along the folding line.

### TECHNICAL FIELD

The following description relates to a retinal electrode device.

### BACKGROUND

A device that is inserted into the eyeball of an organism such as a human or an animal to apply electrical stimulation to the retina has been developed. An exemplary device includes a structure in which an electrode array having a plurality of electrodes for applying electrical stimulation to the retina is installed on a substrate and which controls the electrode array by a controller from the outside of the eyeball. U.S. Patent Application Publication No. 2011/0166623 discloses a form of an implantable retinal electrode array for minimal retinal damage and a method of reducing retinal stress.

### DISCLOSURE OF THE INVENTION

### Technical Goals

An aspect provides a retinal electrode device which minimizes an incision area of an organism upon insertion into the retina and maximizes the range of electrical communication after insertion into the retina.

### Technical Solutions

According to an aspect, there is provided a retinal electrode device including a base, a first wing connected to a first side of the base, a first folding line being defined between the base and the first wing, and a plurality of electrodes installed on at least one or more of the base and the first wing, wherein the retinal electrode device may be configured to take a first form in which the first wing is folded with respect to the base along the folding line and a second form in which the first wing is unfolded with respect to the base along the folding line.

The retinal electrode device may further include a second wing connected to a second side of the base, and a second folding line may be defined between the base and the second wing.

The retinal electrode device may further include a fixing part configured to fix the first wing and the second wing.

When the retinal electrode device takes the second form, the first wing and the second wing may be unfolded simultaneously.

The retinal electrode device may further include a third wing connected to a third side of the base, and a third folding line may be defined between the base and the third wing. When the retinal electrode device takes the first form, the third wing may be folded with respect to the base to be disposed under the first wing, and when the retinal electrode device takes the second form, the third wing may be unfolded with respect to the base after the first wing is unfolded with respect to the base.

The plurality of electrodes may include a plurality of first electrodes of a first group arranged in a first direction and a plurality of second electrodes of a second group arranged in the first direction. When viewed in a second direction intersecting the first direction, the plurality of first electrodes of the first group and the plurality of second electrodes of the second group may not overlap each other.

The first wing may include a first region having a first edge inclined in a first oblique direction with respect to the first side, a second region having a second edge inclined in a second oblique direction with respect to the first side, and a third region having a rounded third edge connecting the first edge and the second edge.

The first wing may include a region defined by a pair of first edges extending perpendicularly to the first side, a second edge extending parallel to the first side, and a pair of rounded third edges connecting the pair of first edges and the second edge.

The retinal electrode device may further include a circuit board installed on the base and configured to control the plurality of electrodes.

The circuit board may be installed only in the center of the base.

The circuit board may cover an entire area of the base, and the plurality of electrodes may be arranged on the circuit board.

According to another aspect, there is provided a retinal electrode device including a base, a first wing connected to a first side of the base, and a plurality of electrodes installed on at least one or more of the base and the first wing, wherein the retinal electrode device may be configured to take a first form in which the first wing is rolled toward the base and a second form in which the first wing is rolled away from the base.

The retinal electrode device may further include a second wing connected to a second side of the base, wherein the second wing may be configured to be rolled toward the base when the retinal electrode device takes the first form, and the second wing may be configured to be rolled away from the base when the retinal electrode device takes the second form.

The retinal electrode device may further include a fixing part configured to fix the first wing and the second wing.

When the retinal electrode device takes the second form, the second wing may be rolled away from the base after the first wing is rolled away from the base.

The retinal electrode device may further include a third wing connected to a third side of the base, wherein the third wing may be configured to be rolled with the first wing in a second direction intersecting a first direction while being unrolled in the first direction toward the base when the retinal electrode device takes the first form.

According to another aspect, there is provided a retinal electrode device including a base and a plurality of electrodes installed on the base, wherein the retinal electrode device is configured to take a first form in which the base is rolled and a second form in which the base is rolled so that a pair of opposing edges of the base move away from each other.

When the retinal electrode device takes the second form, any one edge of the pair of edges may be fixed in place.

The retinal electrode device may further include a fixing part configured to connect a first portion of the base adjacent to a first edge of the pair of edges and a second portion of the base adjacent to a second edge opposite to the first edge.

The base may have a length and a width, and the length of the base may be greater than the width of the base.

### Advantageous Effects

A retinal electrode device according to an example embodiment may minimize the incision area upon insertion into the retina

The retinal electrode device according to an example embodiment may maximize the range of electrical communication.

The retinal electrode device according to an example embodiment may be in close contact with a target site without buckling.

The retinal electrode device according to an example embodiment may improve the density of an electrode array.

Effects of the retinal electrode device according to an example embodiment are not limited to those mentioned above, and other effects not mentioned will be clearly understood by those skilled in the art from the following description.

### BRIEF DESCRIPTION OF DRAWINGS

FIG. 1 is an operation diagram illustrating a retinal electrode device according to an example embodiment.

FIG. 2 is a perspective view illustrating a first form of a retinal electrode device according to a first example embodiment.

FIG. 3 is a perspective view illustrating a second form of the retinal electrode device according to the first example embodiment.

FIG. 4 is a plan view illustrating the retinal electrode device according to the first example embodiment.

FIG. 5 is an enlarged view of part B of FIG. 4.

FIG. 6 is a diagram illustrating conformational changes of the retinal electrode device according to the first example embodiment.

FIG. 7 is a perspective view illustrating a second form of a retinal electrode device according to a second example embodiment.

FIG. 8 is a perspective view illustrating a first form of a retinal electrode device according to a third example embodiment.

FIG. 9 is a perspective view illustrating a second form of the retinal electrode device according to the third example embodiment.

FIG. 10 is a diagram illustrating conformational changes of the retinal electrode device according to the third example embodiment.

FIG. 11 is a perspective view illustrating a second form of a retinal electrode device according to a fourth example embodiment.

FIG. 12 is a perspective view illustrating a first form of a retinal electrode device according to a fifth example embodiment.

FIG. 13 is a perspective view illustrating an intermediate form of the retinal electrode device according to the fifth example embodiment.

FIG. 14 is a perspective view illustrating a second form of the retinal electrode device according to the fifth example embodiment.

FIG. 15 is a perspective view illustrating a first form of a retinal electrode device according to a sixth example embodiment.

FIG. 16 is a perspective view illustrating an intermediate form of the retinal electrode device according to the sixth example embodiment.

FIG. 17 is a perspective view illustrating a second form of the retinal electrode device according to the sixth example embodiment.

FIG. 18 is a diagram illustrating a system including a retinal electrode device according to an example embodiment.

FIG. 19 is a diagram illustrating a system including a retinal electrode device according to another example embodiment.

### BEST MODE FOR CARRYING OUT THE INVENTION

Hereinafter, example embodiments will be described in detail with reference to exemplary drawings. In adding reference numerals to components in each drawing, it should be noted that the same components are given with the same reference numerals as much as possible even though the components are shown in different drawings. In addition, in describing an example embodiment, if it is determined that a detailed description of a related known configuration or function interferes with the understanding of the example embodiment, the detailed description thereof will be omitted.

In addition, in describing the components of an example embodiment, terms such as first, second, A, B, (a), and (b) may be used. These terms are only to distinguish one component from another component, and the nature, sequence, or order of the components is not limited by the terms. When it is mentioned that one component is “connected,” “coupled,” or “accessed” to another component, the component may directly be connected or accessed to the other component, but it should be construed that another component may also be “connected,” “coupled,” or “ accessed” therebetween.

A component included in one example embodiment and components having a common function will be described using the same name in other example embodiments. Unless otherwise stated, a description illustrated in one example embodiment may be applied to another example embodiment, and a detailed description in the overlapping range will be omitted.

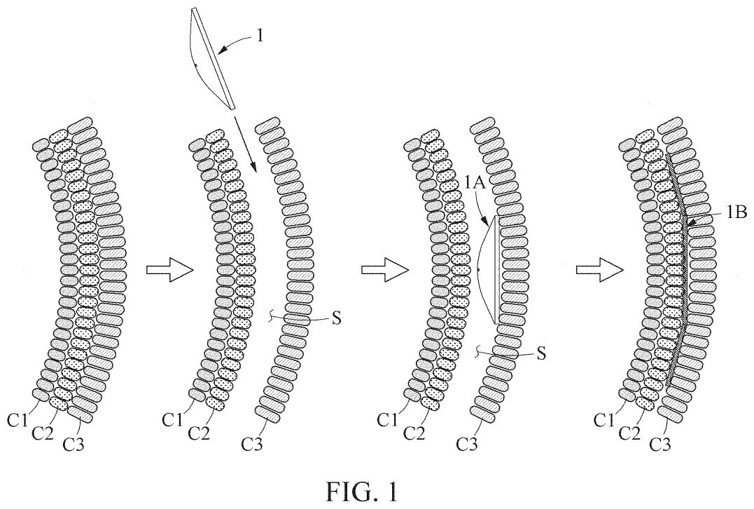

Referring to FIG. 1, a retinal electrode device 1 according to an example embodiment is inserted into the eyeball of a subject to transmit an electrical signal to the retina or detect an electrical signal from the retina. Here, the subject may include an organism such as a human or an animal. For example, the retinal electrode device 1 may be inserted into the sub-retina having a ganglion cell layer C1, a bipolar cell layer C2, and a photoreceptor layer C3 in the eyeball of the subject. If the photoreceptor layer C3 is damaged, the retinal electrode device 1 may be inserted into a space S between the bipolar cell layer C2 and the photoreceptor layer C3.

The retinal electrode device 1 may undergo conformational changes between a first form 1A and a second form 1B. For example, in the space S between the bipolar cell layer C2 and the photoreceptor layer C3, the retinal electrode device 1 may undergo conformational changes from the first form 1A that is reduced to be inserted into the space S to the second form 1B that is expanded for application of electrical stimulation to the retina in the space S. Such conformational changes of the retinal electrode device 1 may minimize the incision area of the subject upon insertion of the retinal electrode device 1 into the retina inside the eyeball, maximize the range of the electrical stimulation during the operation of the retinal electrode device 1, and secure a wide viewing angle. In addition, the retinal electrode device 1 may well adhere to a curved surface with a large curvature such as the retina without folding such as buckling.

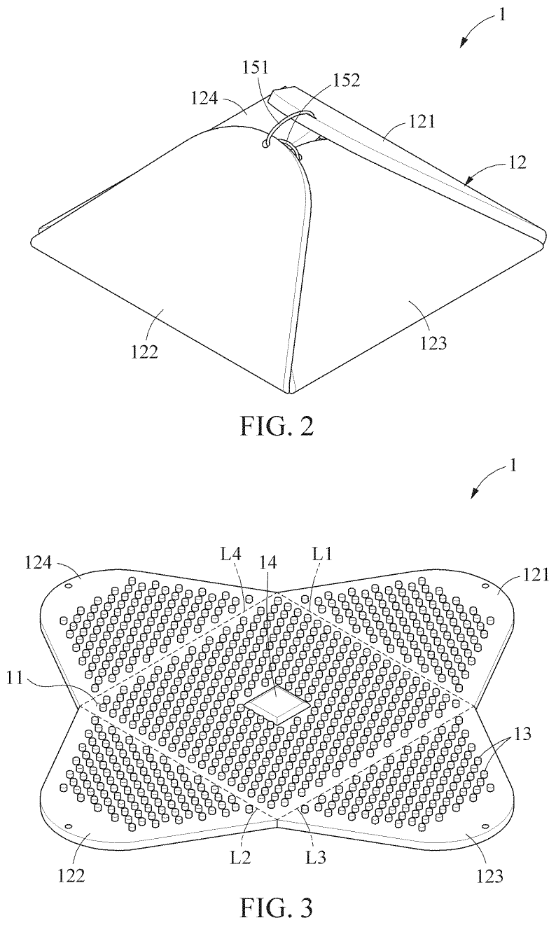

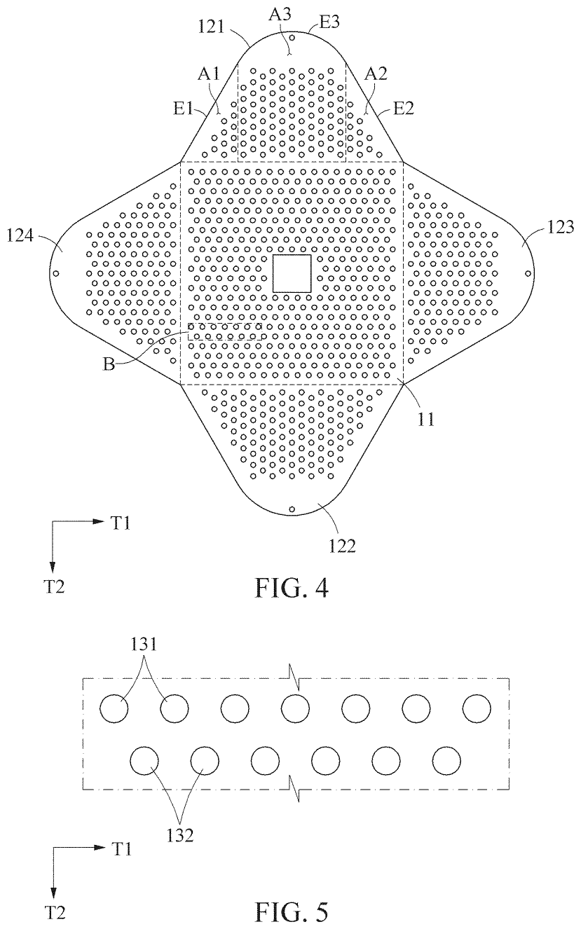

Referring to FIGS. 2 to 5, the retinal electrode device 1 according to an example embodiment may include a base 11, at least one or more wings 12, a plurality of electrodes 13, a circuit board 14, and at least one or more fixing parts 151 and 152.

The base 11 may support at least one or more wings 12. The base 11 may have a substantially flat shape. Also, the base 11 may have a substantially rectangular shape with a plurality of sides. The base 11 may be formed of a flexible material to have flexibility sufficient to adhere to the curved profile. For example, the base 11 may be formed of a silicone-based polymer material.

At least one or more wings 12 may be configured to be folded or unfolded with respect to the base 11. For example, at least one or more wings 12 may include a first wing 121, a second wing 122, a third wing 123, and a fourth wing 124. The first wing 121, the second wing 122, the third wing 123, and the fourth wing 124 may be folded toward the base 11 or unfolded from the base 11. The first wing 121 may be connected to a first side of the base 11, the second wing 122 to a second side opposite to the first side of the base 11, the third wing 123 to a third side between the first side and the second side of the base 11, and the fourth wing 124 to a fourth side opposite to the third side of the base 11. A first folding line L1, which is a reference line for folding or unfolding of the first wing 121, is defined between the first wing 121 and the first side of the base 11, a second folding line L2, which is a reference line for folding or unfolding of the second wing 122, is defined between the second wing 122 and the second side of the base 11, a third folding line L3, which is a reference line for folding or unfolding of the third wing 123, is defined between the third wing 123 and the third side of the base 11, and a fourth folding line L4, which is a reference line for folding or unfolding of the fourth wing 124, is defined between the fourth wing 124 and the fourth side of the base 11.

The first wing 121, the second wing 122, the third wing 123, and the fourth wing 124 may have a substantially triangular shape. Taking the first wing 121 as an example, the first wing 121 may include a first region A1 having a first edge E1 inclined in a first oblique direction with respect to the first side of the base 11, a second region A2 having a second edge E2 inclined in a second oblique direction with respect to the first side of the base 11, and a third region A3 having a third edge E connecting the first edge E1 and the second edge E2 and having a round shape. The shape of the first wing 121 may equally be applied to the shapes of the second wing 122, the third wing 123, and the fourth wing 124.

At least one or more wings 12 may be formed of a flexible material to have flexibility sufficient to adhere to the curved profile. For example, at least one or more wings 12 may be formed of a silicone-based polymer material.

The base 11 and at least one or more wings 12 may be integrally formed to have the same material.

The plurality of electrodes 13 may be configured to generate or record electrical signals. The plurality of electrodes 13 may be formed in the base 11 and/or the inside of at least one or more of the first wing 121, the second wing 122, the third wing 123, and the fourth wing 124.

The plurality of electrodes 13 may be arranged to have a plurality of columns. The plurality of electrodes 13 may include a plurality of first electrodes 131 of a first group arranged in a first direction T1 and a plurality of second electrodes 132 of a second group arranged in the first direction T1, wherein the plurality of first electrodes 131 of the first group and the plurality of second electrodes 132 of the second group may be arranged in parallel to each other. Also, the plurality of first electrodes 131 of the first group and the plurality of second electrodes 132 of the second group may be arranged without overlapping each other when viewed along the second direction T2 intersecting the first direction T1.

The circuit board 14 may control the plurality of electrodes 13. In one example, the circuit board 14 may control the intensity of the electrical signal generated by the plurality of electrodes 13 and the cycle of the electrical signal. In another example, the circuit board 14 may process electrical signals recorded by the plurality of electrodes 13. The size of the circuit board 14 may be smaller than that of the base 11. The circuit board 14 may be installed only in the center of the base 11. The plurality of electrodes 13 may not be installed in the center of the base 11 in which the circuit board 14 is installed.

A structure, to which a controller for controlling the plurality of electrodes 13 is installed on the outside and which has a connector for connecting the controller to the plurality of electrodes 13 of the retinal electrode device 1 in the eyeball of the subject, may cause a bottleneck phenomenon in the connector between the controller outside the subject and the plurality of electrodes 13 inside the subject. In order to solve the issue, it is required to limit the density of the plurality of electrodes 13. In contrast, according to the structure of the retinal electrode device 1 in which the circuit board 14 is directly installed on the base 11, when the retinal electrode device 1 is inserted into the eyeball of the subject, the bottleneck phenomenon of the connector (not shown) connecting the plurality of electrodes 13 and the circuit board 14 does not occur since the controller for controlling the plurality of electrodes 13 is not installed on the outside of the subject remotely from the plurality of electrodes 13. In addition, there is an advantage that the plurality of electrodes 13 may be arranged at a high density in accordance with the structure of the base 11 and at least one or more wings 12.

At least one or more fixing parts 151 and 152 may fix the plurality of wings 121, 122, 123, and 124. The retinal electrode device 1 to which the plurality of wings 121, 122, 123, and 124 are fixed may have the size suitable to be inserted into the eyeball of the subject.

At least one or more fixing parts 151 and 152 may include a first fixing part 151 and a second fixing part 152. The first fixing part 151 may fix the first wing 121 and the second wing 122 facing each other. The second fixing part 152 may fix the third wing 123 and the fourth wing 124 facing each other. For example, the first fixing part 151 may connect the tip of the first wing 121 and the tip of the second wing 122, and the second fixing part 152 may connect the tip of the third wing 123 and the tip of the fourth wing 124.

At least one or more fixing parts 151 and 152 may have a string shape.

In an example embodiment, in the form of the retinal electrode device 1 in which the first wing 121 and the second wing 122, and the third wing 123 and the fourth wing 124 are fixed by the first fixing part 151 and the second fixing part 152, respectively, the third wing 123 and the fourth wing 124 fixed by the second fixing part 152 may be disposed under the first wing 121 and the second wing 122 fixed by the first fixing part 151 while being folded with respect to the base 11. Therefore, when the fixing of the first fixing part 151 and the second fixing part 152 is released, the first wing 121 and the second wing 122 may first be unfolded from the base 11, and the third wing 123 and the fourth wing 124 may be unfolded from the base 11 thereafter.

In an example embodiment not shown, the first wing 121 and the second wing 122 may be disposed under the third wing 123 and the fourth wing 124 while being folded with respect to the base 11.

In another example embodiment not shown, the first wing 121, the second wing 122, the third wing 123, and the fourth wing 124 may be disposed to substantially form a shape of quadrangular pyramid that each distal end is gathered together. In this case, the first wing 121, the second wing 122, the third wing 123, and the fourth wing 124 may be unfolded from the base 11 at the same time when the fixing of the first fixing part 151 and the second fixing part 152 is released.

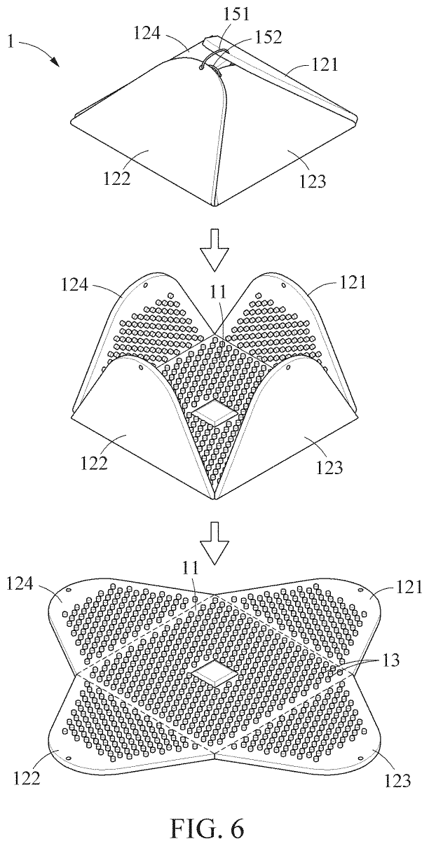

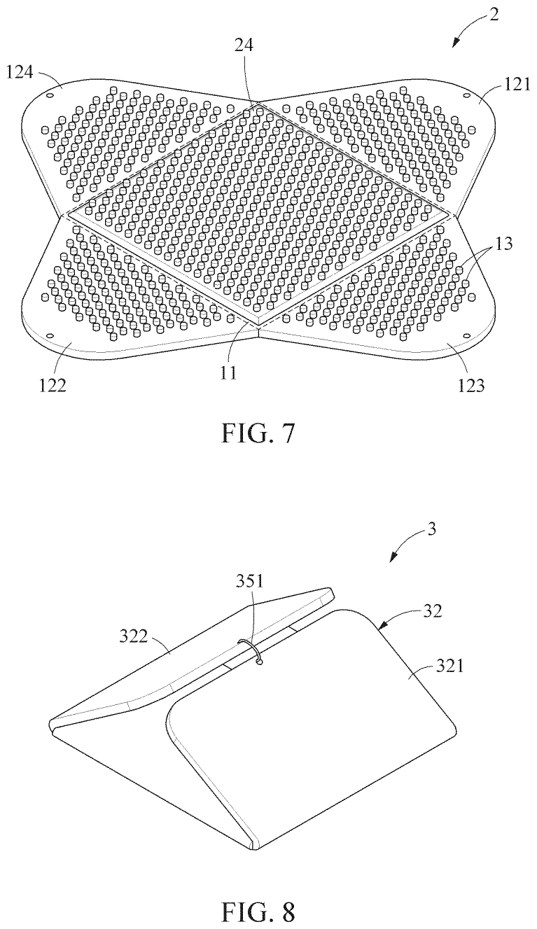

Referring to FIG. 6, according to the first form of the retinal electrode device 1 according to an example embodiment, the first fixing part 151 may fix the first wing 121 and the second wing 122 in a state where the first wing 121 and the second wing 122 are folded with respect to the base 11, and the second fixing part 152 may fix the third wing 123 and the fourth wing 124 in a state where the third wing 123 and the fourth wing 124 are folded with respect to the base 11. Thereafter, when the fixing of the first fixing part 151 and the second fixing part152 is released, the retinal electrode device 1 may take the second form that the first wing 121, the second wing 122, the third wing 123, and the fourth wing 124 are unfolded from the base 11 while the exposure of the plurality of electrodes 13 is involved, wherein the plurality of electrodes 13 is formed in the base 11 and/or at least one or more of the first wing 121, the second wing 122, the third wing 123, and the fourth wing 124.

Referring to FIG. 7, a retinal electrode device 2 according to an example embodiment includes the base 11, the plurality of wings 121, 122, 123, and 124, and the plurality of electrodes 13 which are described above with reference to FIGS. 2 to 5, and may undergo conformational changes of the retinal electrode device 1 described above with reference to FIG. 6. The retinal electrode device 2 of the example embodiment may include a circuit board 24 substantially covering an entire area of the base 11. The circuit board 24 may substantially cover the entire area of the base 11. In this case, a circuit may be formed on the lower surface of the circuit board 24, and the plurality of electrodes 13 and a structure formed of a polymer material may be formed on the upper surface of the circuit board 24.

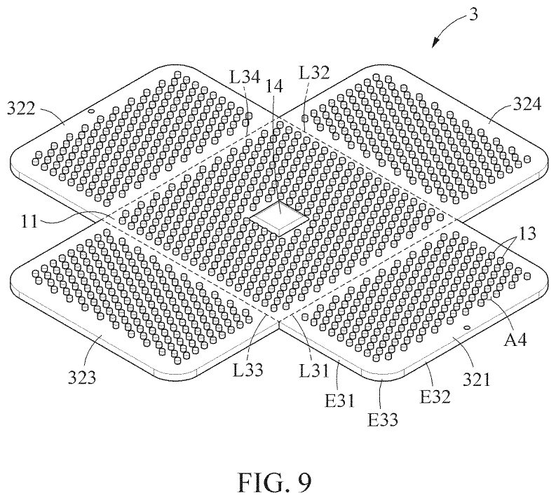

Referring to FIGS. 8 and 9, a retinal electrode device 3 according to an example embodiment includes the base 11, the plurality of electrodes 13, and the circuit board 14 described above with reference to FIGS. 2 to 5, and may include, from a functional point of view, at least one or more wings 32 and at least one or more fixing parts 351 corresponding to the at least one or more wings 12 and the at least one or more fixing parts 151 and 152 described with reference to FIGS. 2 and 5, respectively.

The at least one or more wings 32 may include a first wing 321, a second wing 322, a third wing 323, and a fourth wing 324. The first wing 321 may be connected to the first side of the base 11, the second wing 322 to the second side opposite the first side of the base 11, and the third wing 323 to the third side between the first side and the second side of the base 11, and the fourth wing 324 to the fourth side opposite the third side of the base 11. A first folding line L31, which is a reference line for folding or unfolding of the first wing 321, may be defined between the first wing 321 and the first side of the base 11, a second folding line L32, which is a reference line for folding or unfolding of the second wing 322, may be defined between the second wing 322 and the second side of the base 11, a third folding line L33, which is a reference line for folding or unfolding of the third wing 323, may be defined between the third wing 323 and the third side of the base 11, and a fourth folding line L34, which is a reference line for folding or unfolding of the fourth wing 324, may be defined between the fourth wing 324 and the fourth side of the base 11.

The first wing 321, the second wing 322, the third wing 323, and the fourth wing 324 may have a substantially rectangular shape. Taking the first wing 321 having a region A4 as an example, the region A4 of the first wing 321 may be defined by a pair of first edges E31 extending substantially perpendicular to the first side of the base 11, a second edge E32 extending substantially parallel to the first side of the base 11, and a pair of third edges E33 connecting the pair of first edges E31 and the second edge E32 and having a rounded shape. The shape of the first wing 321 may be equally applied to the shapes of the second wing 322, the third wing 323, and the fourth wing 324.

The at least one or more fixing parts 351 may fix the plurality of wings 321, 322, 323, and 324. In one example, the at least one or more fixing parts 351 may include a single fixing part 351. The single fixing part 351 may fix the first wing 321 and the second wing 322, and may not fix the third wing 323 and the fourth wing 324. In another example not shown, the at least one or more fixing parts 351 may include a plurality of fixing parts 351. Any one of the plurality of fixing parts 351 may fix the first wing 321 and the second wing 322, and another fixing part (not shown) may fix the third wing 323 and the fourth wing 324.

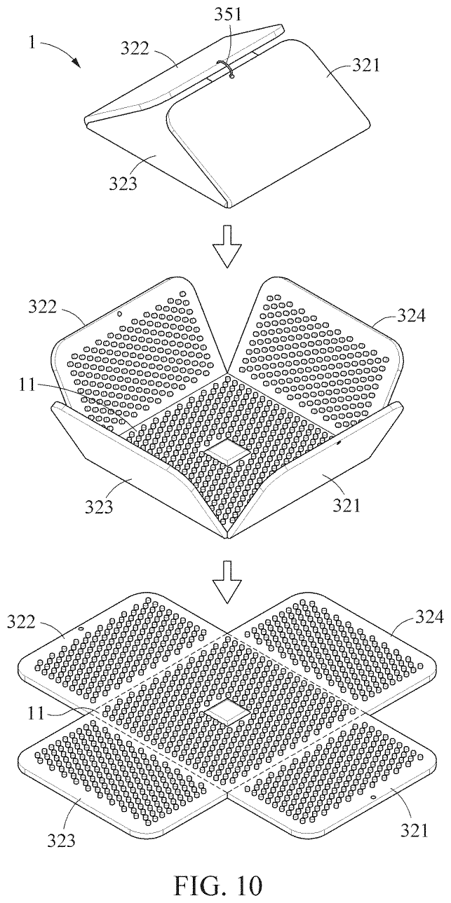

Referring to FIG. 10, according to the first form of a retinal electrode device 3 according to an example embodiment, the first wing 321 and the second wing 322 may be disposed while being fixed by the single fixing part 351 above the third wing 323 and the fourth wing 324 in a state where the third wing 323 and the fourth wing 324 are folded with respect to the base 11. Thereafter, when the fixing of the single fixing part 351 is released, the retinal electrode device 3 may take the second form that the first wing 321 and the second wing 322 are unfolded from the base 11, and then the plurality of electrodes 13 are exposed as the third wing 323 and the fourth wing 324 are unfolded from the base 11.

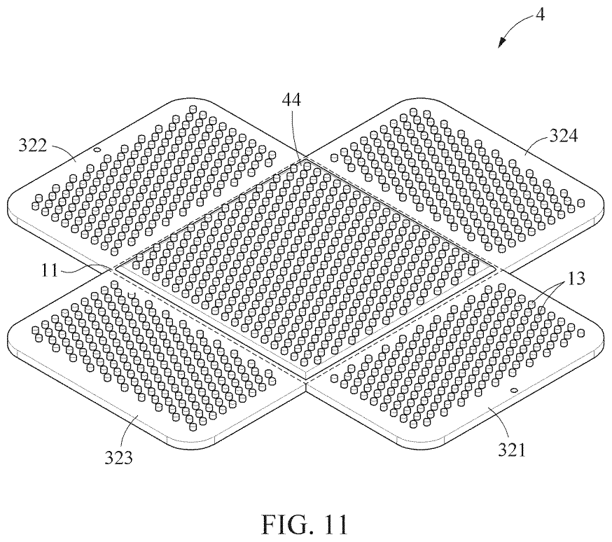

Referring to FIG. 11, a retinal electrode device 4 according to an example embodiment includes the base 11, the plurality of wings 321, 322, 323, and 324, and the plurality of electrodes 13 described above with reference to FIGS. 8 and 9, includes a circuit board 44 corresponding to the circuit board 24 described above with reference to FIG. 7 from a functional and conformational point of view, and may undergo conformational changes of the retinal electrode device 3 described above with reference to FIG. 10. In other words, the circuit board 44 of the example embodiment may substantially cover the entire area of the base 11. In this case, a circuit may be formed on the lower surface of the circuit board 44, and the plurality of electrodes 13 and a structure formed of a polymer material may be formed on the upper surface of the circuit board 44.

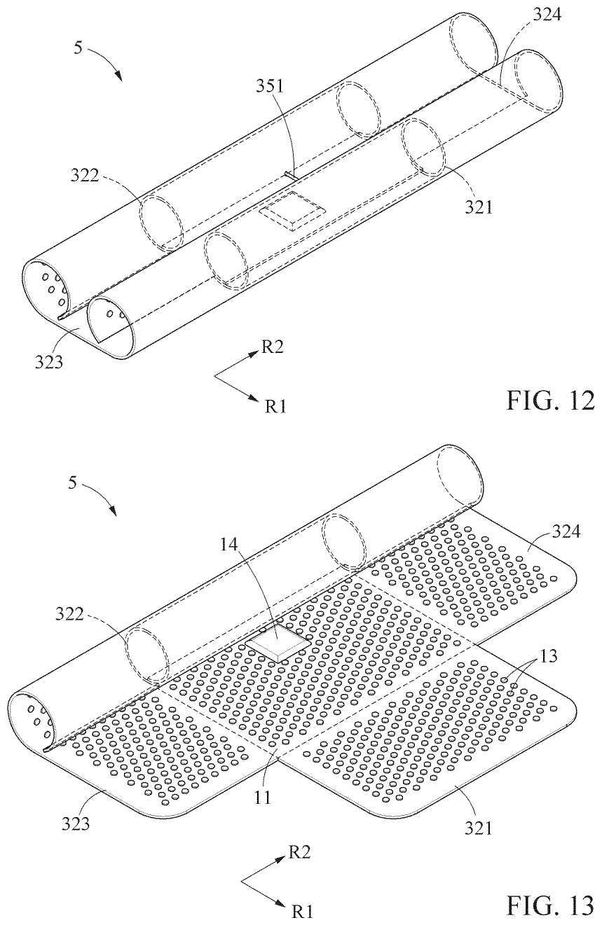

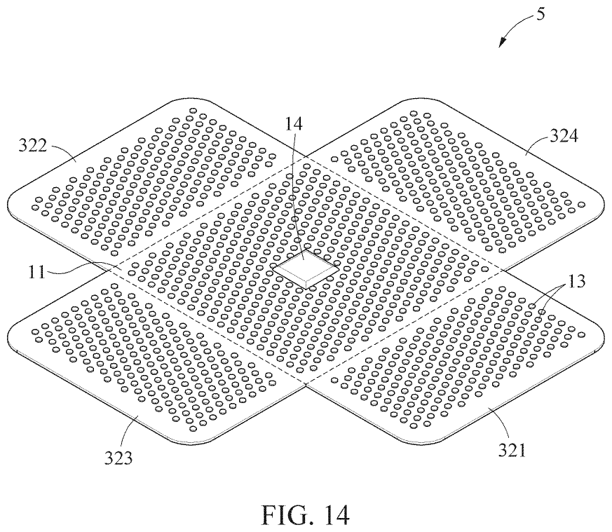

Referring to FIGS. 12 to 14, a retinal electrode device 5 according to an example embodiment includes, from a conformational point of view, the base 11, the plurality of wings 321, 322, 323, and 324, the plurality of electrodes 13, the circuit board 14, and the single fixing part 351 described above with reference to FIGS. 8 and 9.

According to the first form of the retinal electrode device 5, it is possible to maintain a state in which the first wing 321 and the second wing 322 are rolled in the first direction (R1, −R1) toward the base 11 while the third wing 323 and the fourth wing 324 are unrolled in the second direction (R2, −R2) toward the base 11. Here, the first direction (R1, −R1) and the second direction (R2, −R2) may be orthogonal to each other. In the first form of the retinal electrode device 5, the first wing 321 and the second wing 322 may be fixed by the single fixing part 351.

When the fixing of the single fixing part 351 is released, the first wing 321 and the second wing 322 may be rolled in a direction away from the base 11. At this time, at least a portion of the third wing 323 and at least a portion of the fourth wing 324 may also be rolled along with the first wing 321 and the second wing 322 in the same rolling direction of the first wing 321 and the second wing 322. In this case, the rolling of the remaining portions of the first wing 321 and the second wing 322 may be performed after the rolling of the third wing 323 and the fourth wing 324 is completed. On the other hand, when the fixing of the single fixing part 351 is released, the rolling of the first wing 321 may be performed first, and then the rolling of the second wing 322 may be performed.

When the rolling of the first wing 321, the second wing 322, the third wing 323, and the fourth wing 324 is completed in a direction away from the base 11, the retinal electrode device 5 may take the second form that the plurality of electrodes 13 is exposed.

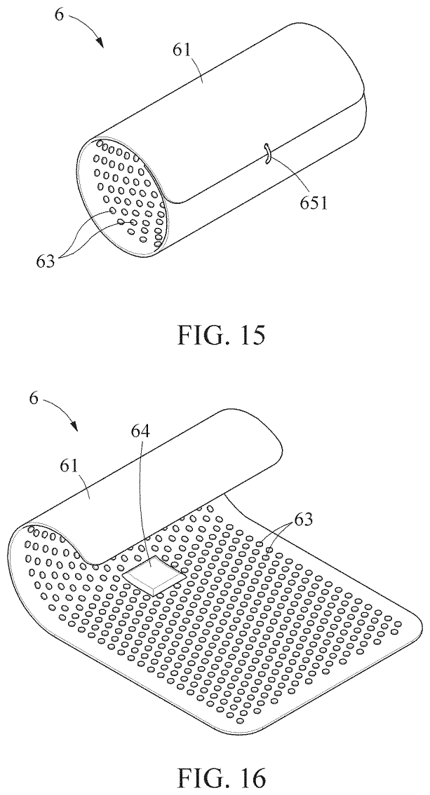

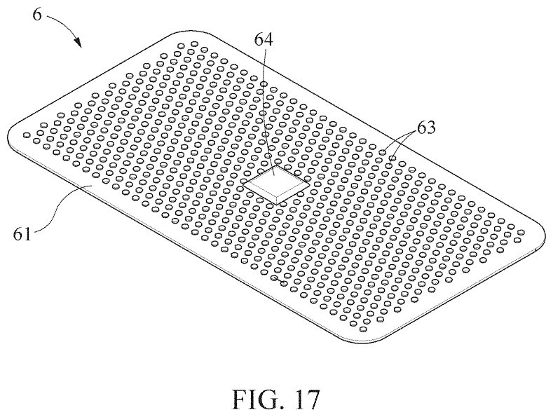

Referring to FIGS. 15 to 17, a retinal electrode device 6 according to an example embodiment may not include a plurality of wings, unlike the example embodiments described above with reference to FIGS. 1 to 14. In other words, the retinal electrode device 6 may include a base 61 excluding a plurality of wings, a plurality of electrodes 63, a circuit board 64, and a fixing part 651. The base 61 may have a length and a width, and the length of the base 61 may be greater than the width of the base 61. Unless otherwise stated, the base 61, the plurality of electrodes 63, the circuit board 64, and the fixing part 651 may correspond to the base, the plurality of electrodes, the circuit board, and the fixing part described above with reference to FIGS. 1 to 14.

The retinal electrode device 6 may take a first form in which the base 61 is rolled and a second form in which the base 61 is rolled so that a pair of opposing edges of the base 61 move away from each other. When used, the retinal electrode device 6 may be configured to undergo conformational changes from the first form to the second form.

When the retinal electrode device 6 takes the first form, the fixing part 651 connects a first portion of the base 61 adjacent to any one first edge in a pair of opposing edges among a plurality of edges of the base 61 and a second portion of the base 61 adjacent to the other second edge so as to maintain the first form.

When the retinal electrode device 6 takes the second form, the base 61 may be rolled so that the other second edge moves away from the first edge while the one first edge in the pair of opposing edges among the plurality of edges of the base 61 remains fixed in place. In other words, when the retinal electrode device 6 takes the second form, the base 61 may be rolled only in one direction.

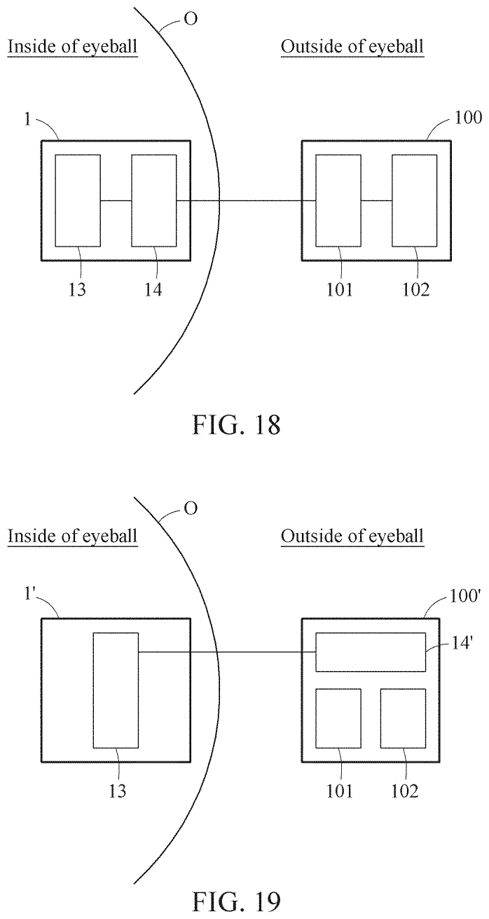

Referring to FIG. 18, a system according to an example embodiment may include the retinal electrode device 1 which includes the plurality of electrodes 13 and the circuit board 14 and a controller 100 which controls the retinal electrode device 1. The controller 100 may include a power/data transmission/reception circuit 101 and a coil 102. The power/data transmission/reception circuit 101 may be configured to be connected to the circuit board 14 and transmit/receive power and/or data to and from the circuit board 14. The coil 102 may transmit and receive electromagnetic signals and supply power to the power/data transmission/reception circuit 101. The retinal electrode device 1 may transmit and receive electrical signals to and from a target region inside the eyeball O while receiving power and/or data from the controller 100.

Referring to FIG. 19, a system according to another example embodiment may include a retinal electrode device 1′ including the plurality of electrodes 13 and a controller 100′ controlling the retinal electrode device 1′. Unlike the example embodiment of FIG. 18, the controller 100′ may include a circuit board 14′ for driving the plurality of electrodes 13. When the power/data transmission/reception circuit 101 and the circuit board 14′ transmit and receive power and/or data, the circuit board 14′ may control the plurality of electrodes 13 of the retinal electrode device 1′.

As described above, although the example embodiments have been described with reference to the limited example embodiments and drawings, various modifications and variations are possible from the above description by one of ordinary skill in the art. For example, suitable results may be achieved if the described techniques are performed in a different order, and/or if components in a described system, architecture, device, or circuit are combined in a different manner and/or replaced or supplemented by other components or their equivalents.

Therefore, other implementations, other example embodiments, and equivalents to the claims are also within the scope of the appended claims.

1. A retinal electrode device comprising:
a base;
a first wing connected to a first side of the base, a first folding line being defined between the base and the first wing; and
a plurality of electrodes installed on at least one or more of the base and the first wing,
wherein the retinal electrode device is configured to take a first form in which the first wing is folded with respect to the base along the folding line and a second form in which the first wing is unfolded with respect to the base along the folding line.

2. The retinal electrode device of claim 1, further comprising:
a second wing connected to a second side of the base, wherein a second folding line is defined between the base and the second wing.

3. The retinal electrode device of claim 2, further comprising:
a fixing part configured to fix the first wing and the second wing.

4. The retinal electrode device of claim 2, wherein the first wing and the second wing are unfolded simultaneously when the retinal electrode device takes the second form.

5. The retinal electrode device of claim 1, further comprising:
a third wing connected to a third side of the base, a third folding line being defined between the base and the third wing,
wherein, when the retinal electrode device takes the first form, the third wing is folded with respect to the base to be disposed under the first wing, and when the retinal electrode device takes the second form, the third wing is unfolded with respect to the base after the first wing is unfolded with respect to the base.

6. The retinal electrode device of claim 1, wherein the plurality of electrodes comprises:
a base;
a first wing connected to a first side of the base; and
a plurality of electrodes installed on at least one or more of the base and the first wing,
wherein the retinal electrode device is configured to take a first form in which the first wing is rolled toward the base and a second form in which the first wing is rolled away from the base.

13. The retinal electrode device of claim 12, further comprising:
a second wing connected to a second side of the base,
wherein the second wing is configured to be rolled toward the base when the retinal electrode device takes the first form, and the second wing is configured to be rolled away from the base when the retinal electrode device takes the second form.

14. The retinal electrode device of claim 13, further comprising:
a fixing part configured to fix the first wing and the second wing.

15. The retinal electrode device of claim 13, wherein the second wing is rolled away from the base after the first wing is rolled away from the base when the retinal electrode device takes the second form.

16. The retinal electrode device of claim 12, further comprising:
a third wing connected to a third side of the base,
wherein the third wing is configured to be rolled with the first wing in a second direction intersecting a first direction while being unrolled in the first direction toward the base when the retinal electrode device takes the first form.

17. A retinal electrode device comprising:
a base; and
a plurality of electrodes installed on the base,
a plurality of first electrodes of a first group arranged in a first direction; and
a plurality of second electrodes of a second group arranged in the first direction, and
when viewed in a second direction intersecting the first direction, the plurality of first electrodes of the first group and the plurality of second electrodes of the second group do not overlap each other.

7. The retinal electrode device of claim 1, wherein the first wing comprises:
a first region having a first edge inclined in a first oblique direction with respect to the first side;
a second region having a second edge inclined in a second oblique direction with respect to the first side; and
a third region having a rounded third edge connecting the first edge and the second edge.

8. The retinal electrode device of claim 1, wherein the first wing comprises a region defined by a pair of first edges extending perpendicularly to the first side, a second edge extending parallel to the first side, and a pair of rounded third edges connecting the pair of first edges and the second edge.

9. The retinal electrode device of claim 1, further comprising:
a circuit board installed on the base and configured to control the plurality of electrodes.

10. The retinal electrode device of claim 9, wherein the circuit board is installed only in the center of the base.

11. The retinal electrode device of claim 9, wherein the circuit board covers an entire area of the base, and the plurality of electrodes are arranged on the circuit board.

12. A retinal electrode device comprising:
wherein the retinal electrode device is configured to take a first form in which the base is rolled and a second form in which the base is rolled so that a pair of opposing edges of the base move away from each other.

18. The retinal electrode device of claim 17, wherein any one edge of the pair of edges is fixed in place when the retinal electrode device takes the second form.

19. The retinal electrode device of claim 17, further comprising:
a fixing part configured to connect a first portion of the base adjacent to a first edge of the pair of edges and a second portion of the base adjacent to a second edge opposite to the first edge.

20. The retinal electrode device of claim 17, wherein the base has a length and a width, and the length of the base is greater than the width of the base.
